(19) United States
(12) Patent Application Publication
(10) Pub. No.: US 2025/0110599 A1
(43) Pub. Date: Apr. 3, 2025

(54) SYSTEMS AND METHODS FOR A MACHINE LEARNING ADAPTABLE USER INTERFACE

(71) Applicant: WELLS FARGO BANK, N.A., San Francisco, CA (US)

(72) Inventors: Michael Asher Cramer, Waimea, HI (US); Samuel B. Martin, San Francisco, CA (US); Eric Kurnie, Woodside, CA (US); Dennis E. Montenegro, Concord, CA (US); Sadie S. Salim, Mill Valley, CA (US)

(21) Appl. No.: 18/478,337

(22) Filed: Sep. 29, 2023

(51) Int. Cl. G06F 3/0481; G06F 9/451; G06N 20/00; G06V 10/774; G06V 40/16; G06V 40/20; G10L 15/06

(52) U.S. Cl. CPC G06F 3/0481; G06F 9/453; G06N 20/00; G06V 10/774; G06V 40/174; G06V 40/20; G10L 15/063

(57) ABSTRACT

Systems and methods for an adaptable user interface based on a user's neurological condition, user experience level, and emotional state including: selecting a neurological condition associated with a user; determining, based on historical user data, a user experience level; generating a user interface based on the selected neurological condition and the user experience level; receiving, from a tracer configured to log a user's activity, a tracking log comprising information regarding the user's activity; determining a behavioral metric by analyzing the tracking log using a machine learning model trained by processing prior user activity, wherein the behavioral metric represents an emotional state of the user; and modifying one or more elements of the user interface based on the behavioral metric.

### FIELD OF INVENTION

The present disclosure generally relates to an artificial intelligence and machine learning system for adapting a user interface to user inputs, and more particularly to systems and methods for using artificial intelligence and machine learning to provide a user interface adapting to a user's neurological condition, user experience, and emotional state.

### BACKGROUND

When designing user interfaces, developers generally cater the user interface to the ability and skill of the average expected user. By setting the user interface to the ability and skill of the average expected user, a user with neurological conditions (e.g., autism, attention deficit hyperactivity disorder, etc.) may be unfairly disadvantaged when operating the user interface because of his or her difference in ability of memory or concentration. Users with neurological conditions may require additional assistance, or additional tools to accomplish the same tasks on a user interface as someone with neurotypical ability. Current user interfaces are also not adaptable to a user's emotional state, such as adapting when the user is frustrated or confused.

### SUMMARY

According to certain embodiments, a method for providing an adaptable user interface for users based on the user's neurological condition, emotional state, and experience is provided. The method includes: selecting a neurological condition associated with a user; determining, based on historical user data, a user experience level; generating a user interface based on the selected neurological condition and the user experience level; receiving, from a tracer configured to log a user's activity, a tracking log comprising information regarding the user's activity; determining a behavioral metric by analyzing the tracking log using a machine learning model trained by processing prior user activity, wherein the behavioral metric represents an emotional state of the user; and modifying one or more elements of the user interface based on the behavioral metric.

In a further embodiment, the neurological condition is selected from a list of neurological conditions comprising one or more of autism, Parkinson's disease, epilepsy, and attention-deficit hyperactivity disorder.

In an additional embodiment, the historical user data includes a measurement of time the user has operated an application.

In another embodiment, the tracking log includes one or more of: mouse movements, mouse hover, periods of mouse inactivity, typing speed, spelling errors, abandoned sessions, and pageviews.

In a further embodiment, the method for providing an adaptable user interface further includes recording a user facial expression; and evaluating the user facial expression, using a machine learning model trained by processing user facial expressions, to update the behavioral metric.

In an additional embodiment, the method for providing an adaptable user interface further includes recording a user's voice; and evaluating the user's voice, using a machine learning model trained by processing user voice for tone, to update the behavioral metric.

In another embodiment, each neurological condition of the list of neurological conditions is associated with a template user interface; and the user interface is generated using the template.

The above methods can be implemented as computer-executable program instructions stored in a non-transitory, tangible computer-readable media and/or operating within a processor or other processing device and memory.

### BRIEF DESCRIPTION OF THE DRAWINGS

FIG. 1 illustrates an example adaptable user interface system communicating between a user device and an application running on cloud service provider (CSP) infrastructure.

FIG. 2 illustrates a flow chart for a method of operating an example adaptable user interface, particularly illustrating adapting the user interface to a user's neurodivergent condition, skill, and emotional state.

FIG. 3 illustrates a flow chart for a method of operating an example adaptable user interface, particularly illustrating use machine learning with a feedback loop of behavioral analysis.

FIG. 4 illustrates a sample list of neurodivergent conditions mapped to user interface templates incremented in functionality and visual representation based on a user's experience and behavioral metrics representing an emotional state.

FIG. 5A and FIG. 5B illustrates a sample user interface transitioning based on a user's experience, neurodivergent condition, and emotional state.

### DETAILED DESCRIPTION

Reference will now be made in detail to various and alternative illustrative examples and to the accompanying drawings. Each example is provided by way of explanation, and not as a limitation. It will be apparent to those skilled in the art that modifications and variations can be made. For instance, features illustrated or described as part of one example may be used in another example to yield a still further example. Thus, it is intended that this disclosure include modifications and variations as come within the scope of the appended claims and their equivalents.

### Illustrative Examples of an Adaptable User Interface System

In one illustrative embodiment, an adaptable user interface system comprises an application for generating a user interface that adapts to a user's neurological condition, experience level, and emotional state. The application receives an input including a selection of a user's neurological condition and uses user inputs from past and current operation of the user interface to determine a user's experience level and a user's emotional state. The system may execute the application on a user device or in cloud service provider infrastructure. Further, in some embodiments, the application may be a microservice.

The user's neurological condition may include various conditions, disorders, and cognitive impairing diseases, such as autism, attention-deficit hyperactivity disorder, or Parkinson's disease. The neurological conditions may further include conditions from the Diagnostic and Statistical Manual of Mental Disorders such as DSM-5-TR or another edition of the manual.

User interfaces include various elements providing visual representations of information and allowing for user input to and navigation through the user interface. For example, elements of the user interface may include input fields, navigational components, and informational components such as visual representations (e.g., graphics, images, visuals) of information.

The adaptable user interface system includes a repository or database including a list of neurological conditions. Each neurological condition is associated with a user interface template. The user interface template may vary in appearance and function based on the neurological condition. For example, the adaptable user interface system may associate a neurological condition of post-traumatic stress disorder with a user interface template including graphics with muted and less vibrant colors. The user interface template may also use slow transitions between moving elements or adjust advertisement permissions of the user interface to prevent potentially triggering content. The user interface may also lower the default volume of actions or remove options from the user interface. In another example, for a user with Parkinson's disease, the user interface may disable motion controls to prevent unintended triggering of motion commands during tremors. The adaptable user interface system may also adjust the size of text font and buttons to allow users lacking precise movement to interact with the user interface more easily.

The adaptable user interface system further adapts the user interface by adding, removing, or modifying functions and changing the appearance of the user interface based on the user's experience level and emotional state. The application may use a tracer, e.g., a tracer application, to track user inputs and a machine learning model to determine a user's experience level and the user's emotional state from the user inputs. For example, the tracer may track the user's mouse movements, mouse hovering, typing speed, typing accuracy, abandoned sessions, and page views. In other examples, the application may use a microphone of a user device to record a user's voice to determine an emotional state of the user from the user's tone, inflections, and choice of words. In further examples, the application may use a camera of the user interface to record a user to determine an emotional state of the user from the user's facial expressions, gesticulations, or body language (e.g., a user putting his or her hands over his or her face, chewing fingernails, or gritting teeth).

For example, the user's emotional state may be upset or frustrated. The adaptable user interface system modifies elements of the user interface based on the user's emotional state, such as by adjusting language used in the user interface. In one such example, when a user is confused, instead stating “Input questions here”, the input field may state “Need help? Ask us a question here.”

The adaptable user interface system further adjusts the user interface based on the experience level of the user. For example, the adaptable user interface system may edit the user interface based on past user inputs and tasks completed by the user on the user interface. As one example, in one embodiment where the user interface is associated with a banking application which the user has previously used to apply for a loan, the application may modify the user interface based on the user's prior application. In such an embodiment, for example, when the user applies for another loan, the adaptable user interface may remove graphics including tutorial steps for applying for the loan. In another embodiment, the application may edit the user interface to include more information regarding the transaction in place of a simplified step by step guide requesting user information.

The adaptable user interface system may use a machine learning model to determine the experience level of the user and the emotional state of the user. The machine learning model may be a pretrained model, pretrained on data mapping user inputs to a set of emotional states and experience levels. In some examples, users may provide feedback to the machine learning model, such as requesting the system add or remove features or elements from the user interface. Based on the user's feedback, the machine learning model may adjust future modifications to the user interface for experience levels and emotional states.

### An Adaptable User Interface System

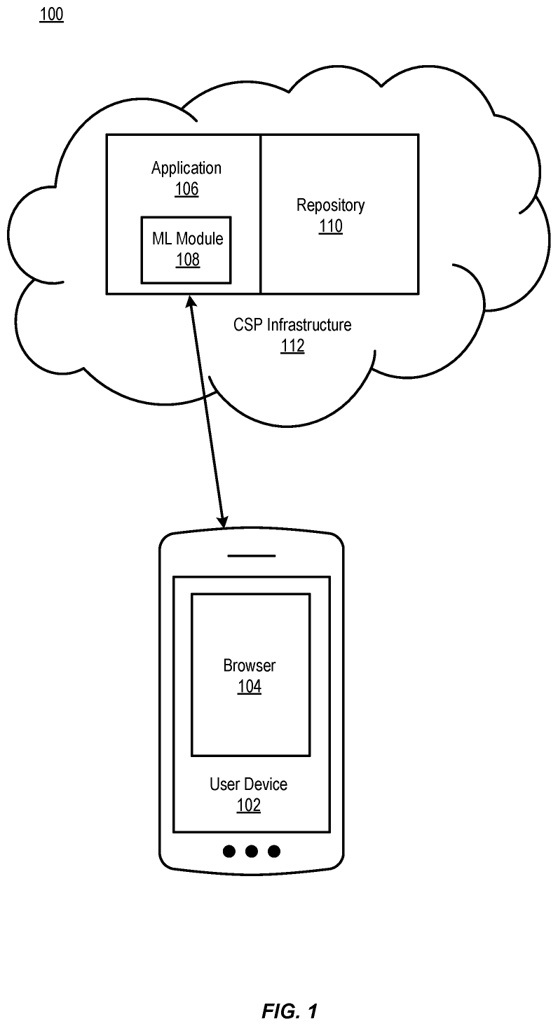

FIG. 1 illustrates an adaptable user interface system 100. The adaptable user interface system includes an application 106 with a machine learning module 108; one or more user devices 102 such as a smartphone, laptop, or tablet executing a browser 104; a repository 110; and cloud service provider (CSP) infrastructure 112, such as various servers, computing devices, processors, and databases.

As shown in FIG. 1, the application 106 may run on the cloud service provider (CSP) infrastructure 112. In other embodiments, the user device 102 may execute the application 106. Users may interact with the application 106 through an adaptable user interface such as a guided user interface (GUI) on a browser 104 or application running locally on the user device 102.

The user device 102 may include various peripherals providing for user input, such as a microphone, a camera, a touch screen, and buttons. For example, the microphone may record inputs such as voice commands and the camera may record facial expressions of the user.

Users may provide further inputs to the application 106 including selecting a neurological condition. Based on the user input, the application 106 queries the repository 110 for a user interface template associated with the selected neurological condition.

The application 106 may download the user interface template from the repository 110 and upload data to repository 110. The application 106 may run on cloud service provider (CSP) infrastructure and comprise various servers and databases to store information associated with user interactions with the application and user interface, such as historical user data. Historical user data includes information associated with the user's past operation of the application, such as past tasks performed by the user within the application, efficiency of the user in performing the past tasks (e.g., how long it takes the user to complete tasks), and the amount of time the user has spent operating the user interface of the application.

In some examples, repository 110 may store user profiles, which may include collections of historical user data associated with the user. The repository 110 may also store information such as user interface templates associated with neurological conditions from the list of neurological conditions. The repository may include a preset mapping of user interface template to one or more neurological conditions. In one embodiment, the adaptable user interface system 100 may associate a first user interface to the neurological conditions of autism and post-traumatic stress disorder, and a second user interface to Parkinson's disease.

In one embodiment, when the application is a finance and banking application, the repository may include a user profile indicating the types of loans that the user has applied for using the application, past questions of the user, the efficiency of the user in performing tasks, and the amount of time the user has spent operating the user interface of the application. The application 106 may query the repository 110 for the historical user data from users.

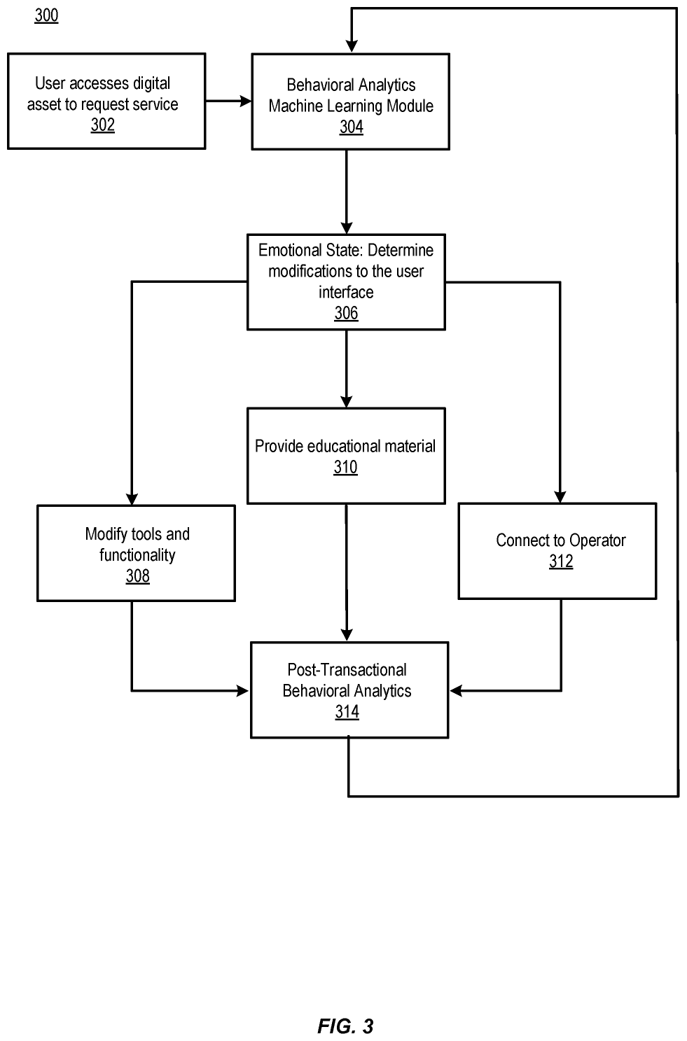

The application 106 may further include a tracer application or program, which may generate a tracking log representing user inputs from a current session of the application 106. The machine learning module 108 may use the tracking log to determine an emotional state of the user and may use the historical user data to determine an experience level of the user. Further description of the machine learning module 108 is provided in the description of FIG. 3.

### Illustrative Methods of Operating an Adaptable User Interface

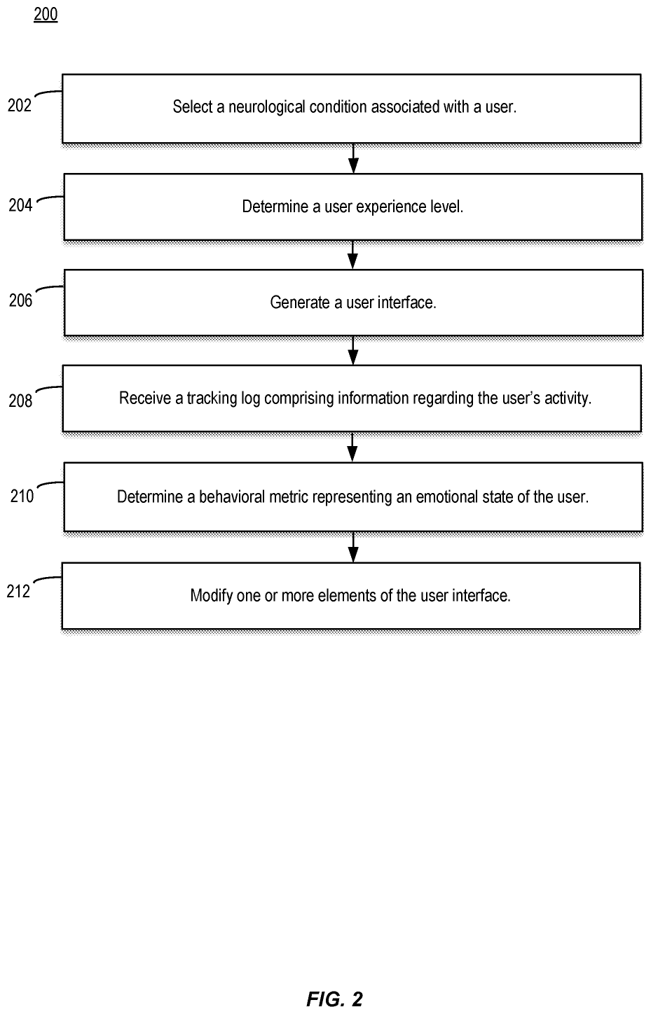

FIG. 2 is a flowchart showing illustrative method 200 for operating an adaptable user interface system. In some embodiments, some of the steps in the flow chart of FIG. 2 are implemented in program code executed by a processor, for example, the processor in a general-purpose computer, mobile device, or server. In some examples, these steps are implemented by a group of processors. In some examples the steps shown in FIG. 2 are performed in a different order or one or more steps may be skipped. Alternatively, in some examples, additional steps not shown in FIG. 2 may be performed.

At step 202, an application, such as application 106 from FIG. 1, selects a neurological condition associated with a user. The application may select the neurological condition from a list of neurological conditions, which may include various neurological conditions such as autism, attention-deficit hyperactivity disorder, Parkinson's disease, epilepsy, Alzheimer's, and post-traumatic stress disorder. The list may further include various neurological conditions from the Diagnostic and Statistical Manual of Mental Disorders (DSM).

The neurological conditions are associated with a user interface template preset to cater to the needs of users with the associated neurological condition. For example, a user interface associated with the neurological condition of epilepsy may include muted colors, removing flashing graphics, and using slower transitions between pages within the user interface (e.g., causing the user interface to transition between pages of the user interface by fading pages in or out instead of using an abrupt transition between pages).

At step 204, the application using a machine learning module, determines a user experience level. The user experience level may be an alphanumeric representation of a user's experience with the application. The machine learning module uses historical user data to determine the user experience level. Historical user data includes information associated with the user's past operation of the application, such as past tasks performed by the user within the application, efficiency of the user in performing the past tasks (e.g., how long it takes the user to complete tasks), and the amount of time the user has spent operating the user interface of the application. For example, historical user data may include information associated with tasks performed by the user within the application and the user's efficiency of performing said tasks. In one such example, user efficiency may include how quickly users complete tasks within the application, which may demonstrate whether the user is able to effectively operate the user interface and application. Historical user data may also include information associated with the amount of time the user has spent within an application, whether the user has requested help, and whether the user has completed a tutorial.

Historical user data may further include past emotional states of the user. For example, past historical user data may indicate that a configuration of the user interface is associated with the user being frustrated. The machine learning module may learn from past emotional states of a user to determine whether modifications should be made to the user interface.

At step 206, the application generates a user interface. The application generates the user interface based on the selected neurological condition and associated user interface template, as well as the user experience level. The application uses the user interface template and adds, removes, or modifies elements and features of the user interface template based on the user experience level.

At step 208, the application receives a tracking log. The tracking log includes information regarding user inputs to the user interface. A tracer program, such as a tracer application, may track the user's inputs to the user interface, and represent the inputs in the tracking log. The tracking log may include various inputs of the user and measured metrics of the user interface, such as movements of the mouse, errors in typing, repeated page views, amount of time spent completing a task, amount of time the user is within the user interface, and incomplete tasks.

At step 210, the machine learning module of the application determines a behavioral metric representing an emotional state of the user based on the tracking log. For example, the tracking log may include information associated with user inputs including mouse movements and viewed pages of the user interface. In one such example, the slow mouse movements and reviewing the same page multiple times may indicate that the user is confused. In another example, rapid clicking and fast mouse movements may indicate the user is frustrated.

At step 212, the application adds, removes, or modifies one or more elements of the user interface based on the behavioral metric. In some examples, the machine learning module from FIG. 1 may determine the user is frustrated and modify elements of the user interface to try to calm the user, such as by asking the user if he or she needs help. By way of example and not to limit the method 200, below is a sample list of behavioral metrics that may be analyzed by the machine learning module when determining a user's emotional state.

Digital Behavioral Metrics
Mouse Movements
Mouse Hover
Mouse Not Moving
Typing Speed
Typing Accuracy
Spelling errors
Abandoned Session
Mouse Speed
Mouse “Jitter”
Input Rate
Scroll Speed
Scroll Pause
Time on Task
Task Success Rate
User Error Rate
Pageviews
Intervention Indicators
Page “Bounce” Rate
Pages per Session
Customer Satisfaction Rates
Customer Experience Quality

### Illustrative Methods of Modifying a User Interface Based on an Emotional State

FIG. 3 is a flowchart showing illustrative method 300 for operating an adaptable user interface system. In some examples, the steps in the flow chart of FIG. 3 are implemented in program code executed by a processor, for example, the processor in a general-purpose computer, mobile device, or server. In some examples, these steps are implemented by a group of processors. In some examples the steps shown in FIG. 3 are performed in a different order or one or more steps may be skipped. Alternatively, in some examples, additional steps not shown in FIG. 3 may be performed.

At step 302, the user may access a digital asset (e.g., the application) to request service. For example, a user may access the application using a user device, such as the user device in FIG. 1, to request a service, such as requesting a loan.

At step 304, the application uses a machine learning module to analyze user inputs. For example, the application may use various machine learning techniques such as neural networks, trained and untrained classifiers, support-vector machines, decision trees, and Bayesian networks to analyze user inputs to determine the user's emotional state. In one example, user inputs may be assigned numerical weights to indicate that the user input is associated with various emotional states. The machine learning module may weigh a combination of user inputs to determine a likely emotional state of the user.

At step 306, the machine learning module determines, based on the emotional state of the user, modifications to the user interface to improve the user's experience. The adaptable user interface system may include preset modifications to be implemented for emotional states. For example, when the adaptable user interface system identifies the user is frustrated, a preset modification may include a popup message asking for the user's feedback. In other examples, the machine learning module may determine the modification. In such an example, the machine learning module may identify that the user is frustrated, and that the user's frustration is associated with a particular element, such as by identifying the user has repeatedly and rapidly clicked an element of the user interface. The machine learning module may identify the element as being a source of the user's frustration and modify the element such providing a pop-up box explaining the element or simplifying the element by removing information provided by the element.

Modifications to the user interface may include the individual modifications and solutions represented in steps 308, 310, and 312 or various combinations thereof.

Step 308 includes modifications to tools and functionality of the user interface. For example, the machine learning module may determine to add, remove, or modify individual tools and functions of the user interface, such as by adjusting the arrangement of elements of a user interface, and adjusting the elements present within the user interface.

Step 310 includes providing educational materials. The educational materials may include tutorials for operating the user interface, or educational materials for tasks the user is attempting to complete on the user interface. For example, when the task is completing a loan application, the educational materials may include an explanation of credit scores, or a warning to the user of actions that may affect approval of a loan.

Step 312 includes connecting the user to an operator, such as by starting an instant message conversation, or a phone call between the user and an operator of the application (e.g., information technology staff or customer service department associated with the adjustable user interface system).

When the modifications or responses to the user's emotional state have been executed or implemented, the machine learning module may analyze the user inputs to determine whether the modifications or responses were effective in assisting the user operate the user interface, such as by identifying whether the user completed a task he or she was working on, or whether the user's efficiency in operating the user interface improved. The analysis of the user inputs after implementing the modification or response is represented at step 314 as post-transactional behavioral analytics. The post-transactional behavioral analytics are then used in a feedback loop to improve the machine learning module's analysis at step 304.

### Illustrative Example of User Interface Templates

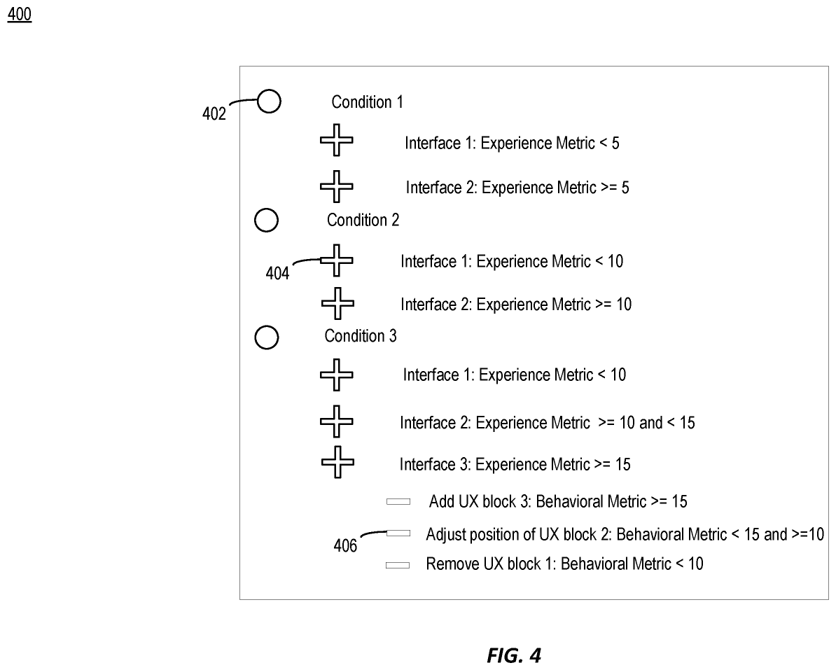

FIG. 4 illustrates an example of logic used by the machine learning module of the adjustable user interface system in determining modifications to the user interface. The adjustable user interface system includes a user interface template for conditions of the user 402. The conditions of the user 402 includes various neurological conditions, such as autism, post-traumatic stress disorder, and attention deficit hyperactivity disorder.

The templates for the conditions of the user are adjustable based on user experience and user's emotional state, represented by an experience metric 404 and behavioral metric 406 respectively. FIG. 4 represents the experience metric 404 and the behavioral metric 406 as numerical values. For example, a machine learning module, such as the machine learning module from FIG. 1, may determine a numerical value for the experience metric based on historical user data and a numerical value for the behavioral metric based on user inputs during operation of the user interface.

### Illustrative User Interface

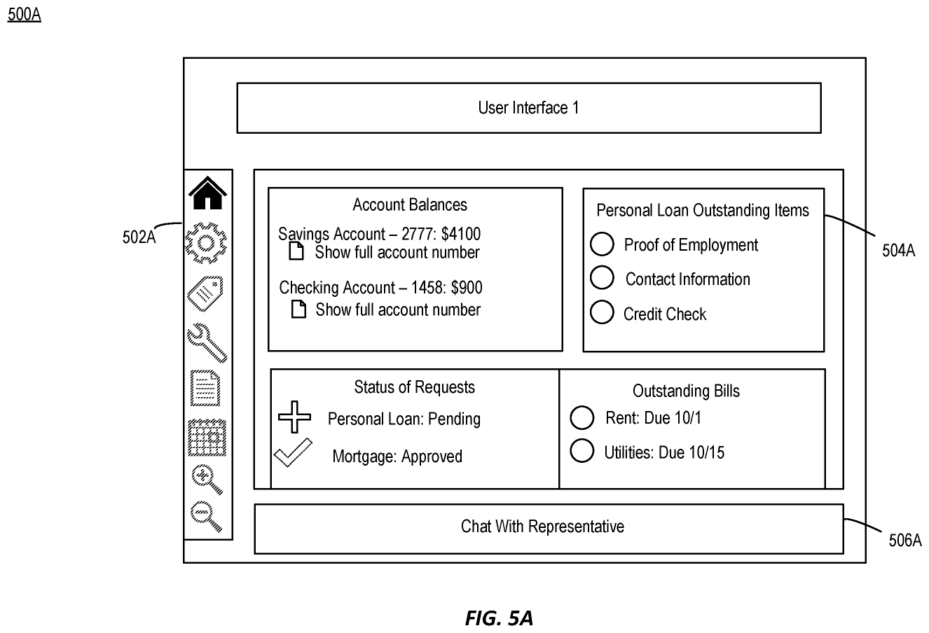

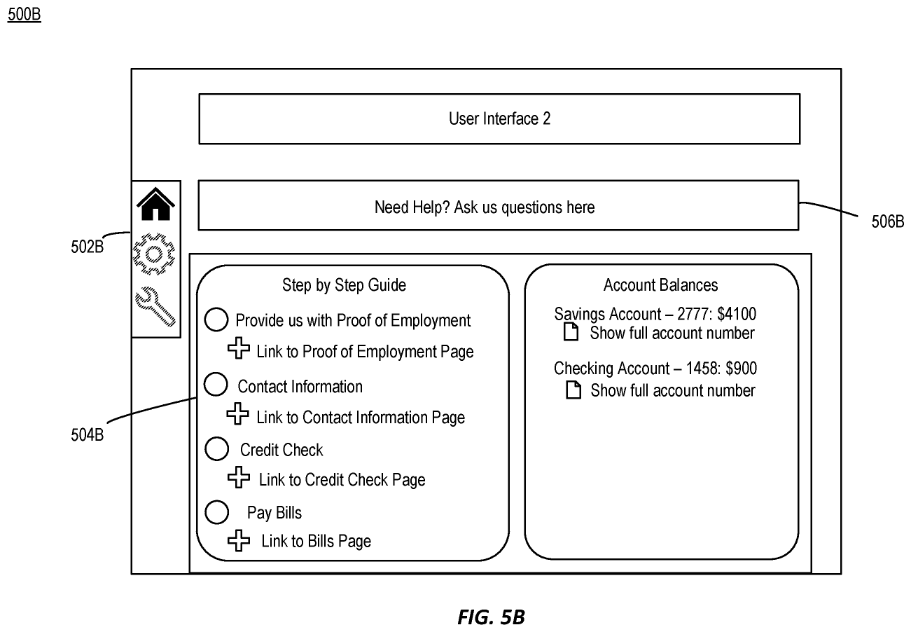

FIG. 5A and FIG. 5B illustrates an example adaptable user interface system transitioning from user interface 1 500A to user interface 2 500B based on a user's emotional state and experience level. User interface 1 500A and user interface 2 500B include a toolbar 502A 502B, graphic 504A 504B, and an input field 506A 506B respectively. User interface 1 500A may be generated from a user interface template, such as the user interface template stored in repository 110 of FIG. 1. By way of example, FIG. 5B illustrates a modified user interface for a frustrated user lacking experience with user interface 1 500A from FIG. 5A.

In such example, the adaptable user interface system may simplify the toolbar 502A and graphic 504A by removing features and providing informational graphics such as a step-by-step guide to cater to the user's lack of experience. For example, toolbar 502A and 502B may represent available functions of user interface 1 500A and user interface 2 500B. The adaptable user interface system may generate a user interface with fewer features, such as the toolbar 502B for users with less experience operating the user interface, as demonstrated in FIG. 5B. The adaptable user interface system may also generate a user interface with simpler graphics, fewer graphics, or additional informational graphics such as graphic 504B, which may include step by step guides, hyperlinks to additional information associated with a graphic, or other informational help to assist users in completing a task and operating the user interface.

User interface 2 500B also includes a modified arrangement of the elements from user interface 1 500B. For example, the input field 506B is in an updated position, moved from the bottom of user interface 1 500A to the top of user interface 2 500B to improve the visibility of the input field 506B.

The adaptable user interface system also modifies user interface 1 500A based on the emotional state of the user. For example, user interface 2 500B includes input field 506B with an updated message catered to a frustrated user by updating the language from “Chat With Representative” to “Need Help? Ask us questions here”. In further examples, modifications to user interface 1 500A may include adding pop up graphics asking the user if the user would prefer a human operator or causing a human operator to contact the user via instant messaging or through a phone call.

### Example Advantages of an Adaptable User Interface System

The adaptable user interface system may provide a dynamic user interface that adjusts based on a user's neurological condition, experience, and emotional state. By generating user interfaces based on the user's neurological condition and modifying the user interface based on the user's experience and emotional state, the adaptable user interface system provides users with a user interface tailored to a user's need that also changes with the user as the user changes. Because the user interface is tailored to the user, the adaptable user interface system provides the user with a user interface the user is better suited to operate efficiently, and a user interface better suited to convey information to users with neurological conditions.

### General Considerations

Although the subject matter has been described in language specific to structural features or methodological acts, it is to be understood that the subject matter of the appended claims is not necessarily limited to the specific features or acts described above. Rather, the specific features and acts described above are disclosed as examples.

Various operations of examples are provided herein. The order in which one or more or all of the operations are described should not be construed as to imply that these operations are necessarily order dependent. Alternative ordering will be appreciated based on this description. Further, not all operations may necessarily be present in each example provided herein.

As used in this application, “or” is intended to mean an inclusive “or” rather than an exclusive “or.” Further, an inclusive “or” may include any combination thereof (e.g., A, B, or any combination thereof). In addition, “a” and “an” as used in this application are generally construed to mean “one or more” unless specified otherwise or clear from context to be directed to a singular form. Additionally, at least one of A and B and/or the like generally means A or B or both A and B. Further, to the extent that “includes”, “having”, “has,” “with,” or variants thereof are used in either the detailed description or the claims, such terms are intended to be inclusive in a manner similar to the term “comprising”.

Further, unless specified otherwise, “first,” “second,” or the like are not intended to imply a temporal aspect, a spatial aspect, or an ordering. Rather, such terms are merely used as identifiers, names, for features, elements, or items. For example, a first state and a second state generally correspond to state 1 and state 2 or two different or two identical states or the same state. Additionally, “comprising,” “comprises,” “including,” “includes,” or the like generally means comprising or including.

Although the disclosure has been shown and described with respect to one or more implementations, equivalent alterations and modifications will occur based on a reading and understanding of this specification and the drawings. The disclosure includes all such modifications and alterations and is limited only by the scope of the following claims.

What is claimed is:

1. A method comprising:
selecting a neurological condition associated with a user;
determining, based on historical user data, a user experience level;
generating a user interface based on the selected neurological condition and the user experience level;
receiving, from a tracer configured to log a user's activity, a tracking log comprising information regarding the user's activity;
determining a behavioral metric by analyzing the tracking log using a machine learning model trained by processing prior user activity, wherein the behavioral metric represents an emotional state of the user; and
modifying one or more elements of the user interface based on the behavioral metric.

2. The method of claim 1, wherein the neurological condition is selected from a list of neurological conditions comprising one or more of autism, Parkinson's disease, epilepsy, and attention-deficit hyperactivity disorder.

3. The method of claim 1, wherein the historical user data includes a measurement of time the user has operated an application.

4. The method of claim 1, wherein the tracking log includes one or more of: mouse movements, mouse hover, periods of mouse inactivity, typing speed, spelling errors, abandoned sessions, and pageviews.

5. The method of claim 1, further comprising:
recording a user facial expression; and
evaluating the user facial expression, using a machine learning model trained by processing prior user facial expressions, to update the behavioral metric.

6. The method of claim 1, further comprising:
recording a user's voice; and
evaluating the user's voice, using a machine learning model trained by processing prior user voice interactions, to update the behavioral metric.

7. The method of claim 2, wherein each neurological condition of the list of neurological conditions is associated with a template user interface; and
wherein the user interface is generated using the template.

8. A system comprising:
a non-transitory computer-readable medium storing computer-executable program instructions; and
a processor communicatively coupled to the non-transitory computer-readable medium for executing the computer-executable program instructions, wherein executing the computer-executable program instructions configures the processor to perform operations comprising:
selecting a neurological condition associated with a user;
determining, based on historical user data, a user experience level;
generating a user interface based on the selected neurological condition and the user experience level;
receiving, from a tracer configured to log a user's activity, a tracking log comprising information regarding the user's activity;
determining a behavioral metric by analyzing the tracking log using a machine learning model trained by processing prior user activity, wherein the behavioral metric represents an emotional state of the user; and
modifying one or more elements of the user interface based on the behavioral metric.

9. The system of claim 8, wherein the neurological condition is selected from a list of neurological conditions comprising one or more of autism, Parkinson's disease, epilepsy, and attention-deficit hyperactivity disorder.

10. The system of claim 8, wherein the historical user data includes a measurement of time the user has operated an application.

11. The system of claim 8, wherein the tracking log includes one or more of: mouse movements, mouse hover, periods of mouse inactivity, typing speed, spelling errors, abandoned sessions, and pageviews.

12. The system of claim 8, further comprising:
recording a user facial expression; and
evaluating the user facial expression, using a machine learning model trained by processing prior user facial expressions, to update the behavioral metric.

13. The system of claim 8, further comprising:
recording a user's voice; and
evaluating the user's voice, using a machine learning model trained by processing prior user voice interactions, to update the behavioral metric.

14. The system of claim 9, wherein each neurological condition of the list of neurological conditions is associated with a template user interface; and
wherein the user interface is generated using the template.

15. A non-transitory computer-readable storage medium storing computer-executable program instructions, wherein when executed by a processor, the computer-executable program instructions cause the processor to perform operations comprising:
selecting a neurological condition associated with a user;
determining, based on historical user data, a user experience level;
generating a user interface based on the selected neurological condition and the user experience level;
receiving, from a tracer configured to log a user's activity, a tracking log comprising information regarding the user's activity;
determining a behavioral metric by analyzing the tracking log using a machine learning model trained by processing prior user activity, wherein the behavioral metric represents an emotional state of the user; and
modifying one or more elements of the user interface based on the behavioral metric.

16. The non-transitory computer-readable storage medium of claim 15, wherein the neurological condition is selected from a list of neurological conditions comprising one or more of autism, Parkinson's disease, epilepsy, and attention-deficit hyperactivity disorder.

17. The non-transitory computer-readable storage medium of claim 15, wherein the historical user data includes a measurement of time the user has operated an application.

18. The non-transitory computer-readable storage medium of claim 15, wherein the tracking log includes one or more of: mouse movements, mouse hover, periods of mouse inactivity, typing speed, spelling errors, abandoned sessions, and pageviews.

19. The non-transitory computer-readable storage medium of claim 15, further comprising:
recording a user facial expression; and
evaluating the user facial expression, using a machine learning model trained by processing prior user facial expressions, to update the behavioral metric.

20. The non-transitory computer-readable storage medium of claim 15, further comprising:
recording a user's voice; and
evaluating the user's voice, using a machine learning model trained by processing prior user voice interactions, to update the behavioral metric.
